(19) United States
(12) Patent Application Publication
(10) Pub. No.: US 2020/0155338 A1
(43) Pub. Date: May 21, 2020

(54) PROSTHETIC STOMA DEVICE AND METHOD OF APPLICATION

(71) Applicant: Karla K. Meteer, Blue Mound, IL (US)

(72) Inventor: Karla K. Meteer, Blue Mound, IL (US)

(21) Appl. No.: 16/688,777

(22) Filed: Nov. 19, 2019

Related U.S. Application Data
(60) Provisional application No. 62/769,199, filed on Nov. 19, 2018.

(51) Int. Cl. A61F 5/448

(52) U.S. Cl. CPC A61F 5/448

(57) ABSTRACT

Embodiments of the invention provide a prosthetic stoma in use with ostomy applications. Particularly, the embodiments disclosed herein provide a prosthetic stoma for insertion into the stoma and ostomy of a patient to provide a more seamless passage for bodily fluids to leave the body. One end of the prosthetic stoma is placed inside of the stoma, while the other end secures the prosthetic stoma to the chosen ostomy device. After application to a patient, the prosthetic stoma decreases or eliminates leakage that can occur when bodily fluids enter an ostomy bag through the stoma.

### CROSS REFERENCE TO RELATED APPLICATIONS

This application claims priority to U.S. Provisional Patent Application No. 62/769,199, filed on Nov. 19, 2018, the entire contents of which is incorporated herein by reference.

### BACKGROUND

In the medical field, a stoma is generally considered any opening in the body, but more frequently refers to an artificial opening in a person's abdominal wall. As a result, stomas can be natural (e.g., a person's mouth) or can be artificially produced by incisions for medical procedures (e.g., an incision for a colostomy). An ostomy is an artificial opening of an organ of the body. Usually, an ostomy is connected to an opening in the abdominal wall (i.e., a stoma) of a patient to create a new path for waste to leave the body. Situations regularly arise that require the use of both an ostomy and a stoma for patients who have one or more of the following conditions: cancer, trauma, bowel obstructions, infections, inflammatory bowel disease, fecal incontinence, or diverticulitis.

An ostomy can be temporary or permanent, depending on the patient's need. For example, when surgery is performed on areas downstream of the small intestine, a temporary ostomy may be required to provide an alternative route for waste to exit the body. However, an ostomy may also be permanent. Permanent ostomies and stomas may be required, for example, for patients with a disease that impairs the normal function of the intestine or the normal function of muscles that are in control of removing waste from the body. When such a situation arises, one or both of an ostomy and/or a stoma may be used for permanent removal of bodily waste.

When an ostomy is created for waste removal purposes, a patient's bodily waste will exit through the ostomy to the stoma, and ultimately out to a bag or pouch. In such a situation, the bag or pouch is usually installed on the outside of the patient's body and is connected to the stoma to collect the bodily waste for subsequent disposal thereof. Usually, the bag that receives the waste is attached to the stoma or to the patient's skin using an adhesive or a device having adhesive applied thereto.

Current pouching systems enable those who require a permanent ostomy to continue everyday activities generally without inhibition. However, complications can arise, including, for example, one of more of stoma retraction, stoma stenosis, stoma trauma, and/or peristomal skin conditions. Additionally, many of the abovementioned complications occur due to leakage of the bodily waste as the bodily waste exits the ostomy through the stoma and into the ostomy bag. The leakage can be a result of a poor seal around the stoma, an ineffective path through which bodily waste can transfer through that may cause damage to the user's skin, or some other reason. Therefore, a more effective path between the ostomy and the ostomy bag is desired to help bodily waste exit the body with a limited amount of leakage therefrom.

### SUMMARY

Some embodiments provide a prosthetic stoma for use with patients who have undergone ostomy and/or stoma medical procedures. In some forms, the prosthetic stoma device can be used with common ostomy devices in use today. The prosthetic stoma has a first opening with a peripheral wall designed to receive an artificial stoma. The first opening includes an entrance for bodily waste to enter the prosthetic stoma. The peripheral wall of the first opening is connected to a tapered portion, which tapers toward a cylindrical shaft having a hollow interior. The cylindrical shaft is connected to a top dome fixture, which is designed to be disposed outside of the body. A second opening is present atop the dome fixture and provides an exit for bodily waste from the prosthetic stoma. The dome fixture has a flange portion extending outward from the base of the dome fixture, which provides a surface for application of an adhesive device. A hollow passage extends between the first and second openings. Thus, bodily waste from the ostomy of a patient may enter and pass through the prosthetic stoma into an ostomy bag for subsequent disposal. As a result, the prosthetic stoma allows for seamless movement of bodily waste from the ostomy and to the ostomy bag without leakage.

In some embodiments, the prosthetic stoma is made from elastically deformable or flexible medical grade silicone, so as to provide a tight seal between the first opening of the prosthetic stoma and the ostomy and/or stoma of the user. Moreover, the elastically deformable or flexible medical grade silicone can provide a tight seal between the cylindrical shaft and the stoma to prevent leakage. It should be appreciated that other materials may be used to create the prosthetic stoma, which provide a tight seal without causing unintended or undesirable consequences to the user thereof.

### DESCRIPTION OF THE DRAWINGS

FIG. 1 is a schematic perspective view of an individual with an ostomy device, an ostomy bag, and a prosthetic stoma according to one embodiment of the invention;

FIG. 2 is sectional view of the ostomy device, the ostomy bag, and the prosthetic stoma, shown in FIG. 1;

FIG. 3 is a side view of the prosthetic stoma shown in FIG. 1; and

FIG. 4 is a front view of the prosthetic stoma shown in FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

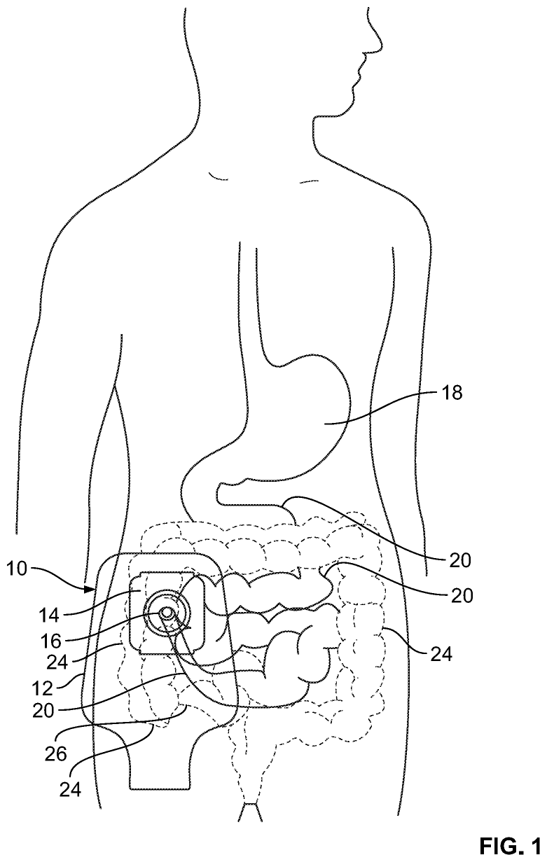

The device of the present disclosure is intended for patients who have undergone an ostomy medical procedure, as many such patients have a pouch or bag attached to their abdominal wall for the collection of bodily waste. Additionally, the embodiments disclosed herein are intended for patients having an artificially incised stoma for bodily waste to exit the body. For example, FIG. 1 illustrates an ostomy device assembly 10 that includes an ostomy bag 12, an adhesive 14, and the prosthetic stoma 16. In this embodiment, the ostomy device assembly 10 is used to provide an exit and storage system for bodily waste. Food or liquid enters the body, moves through the stomach 18 and the small intestine 20, where the food and/or liquid is broken down, and exits the body into the ostomy device assembly 10, where it is stored for later disposal. It is noted that the dashed lines in FIG. 1 illustrate the normal passageway for bodily fluids, i.e., through the patient's intestines. Without the use of the ostomy device assembly 10, the small intestine 20 is connected to the large intestine 24 through a passage 26, where bodily fluids would later exit the body in a normal fashion. The prosthetic stoma 16 can be used in the ostomy device assembly 10 of FIG. 1 and/or the prosthetic stoma 16 may be used in a variety of other medical procedures involving an ostomy, which may include, a colostomy, an ileostomy, a urostomy, and the like.

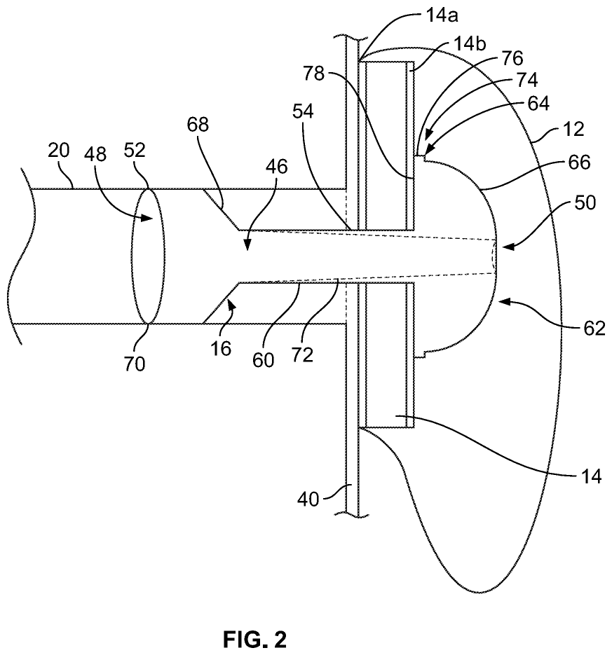

FIG. 2 depicts the ostomy device assembly 10 of FIG. 1 including the prosthetic stoma 16. In this particular embodiment, the ostomy device assembly 10 includes one or more of the prosthetic stoma 16, the adhesive 14, and the ostomy bag 12. The prosthetic stoma 16 is provided with a first opening 48, which allows bodily waste to move from the small intestine 20 into the prosthetic stoma 16. As a result, bodily waste can move through the prosthetic stoma 16 by entering the first opening 48, moving through a passage 46 that connects the first opening with a second opening 50, and exiting through the second opening 50, where the waste enters the ostomy bag 12. The first opening 48 is designed to contact and/or be in communication with a portion of the patient's body (i.e., intestine 20), whereas the second opening 50 is in communication with the ostomy bag 12 or some other waste collection device. In this way, the prosthetic stoma 16 creates a fluid path from inside of the patient's body to the exterior of the patient's body.

In this embodiment, the prosthetic stoma 16 is produced as a one-piece component. However, it should be appreciated that the prosthetic stoma 16 may comprise multiple components, which may be attached to one another to form the prosthetic stoma 16. The prosthetic stoma 16 may be detached and attached to simplify the insertion and/or removal of the prosthetic stoma 16 from the ostomy applications.

Still referring to FIG. 2, the first opening 48 is circular in shape and is defined by a peripheral wall 52. To utilize the assembly 10 with a patient, the first opening 48 and the peripheral wall 52 of the prosthetic stoma 16 is inserted into the patient's stoma and/or ostomy. In one embodiment, the peripheral wall 52 is made from an elastically deformable material or flexible medical grade silicone. Thus, the peripheral wall 52 has a flush and tight engagement with the inside of the small intestine 20 and/or an artificial stoma 54. A circumference of the peripheral wall 52 may be defined by the size of the inside of the small intestine 20 or the associated port or opening of the artificial stoma 54. As a result, the prosthetic stoma 16 provides a seamless passage for the bodily waste to move from the small intestine 20 into the prosthetic stoma 16. It should be appreciated that other materials could be used to create the prosthetic stoma 16, as long as such materials provide the desired fluid-tight fit with the small intestine 20 and/or artificial stoma 54.

The first opening 48 and its peripheral wall 52 may be provided in different sizes or may be substantially the same size. In some embodiments, the peripheral wall 52 may be sized and unique to a patient or user thereof. For example, the peripheral wall 52 may be sized and dimensioned to have a fluid-tight seal with the artificial stoma 54 and/or ostomy of the patient.

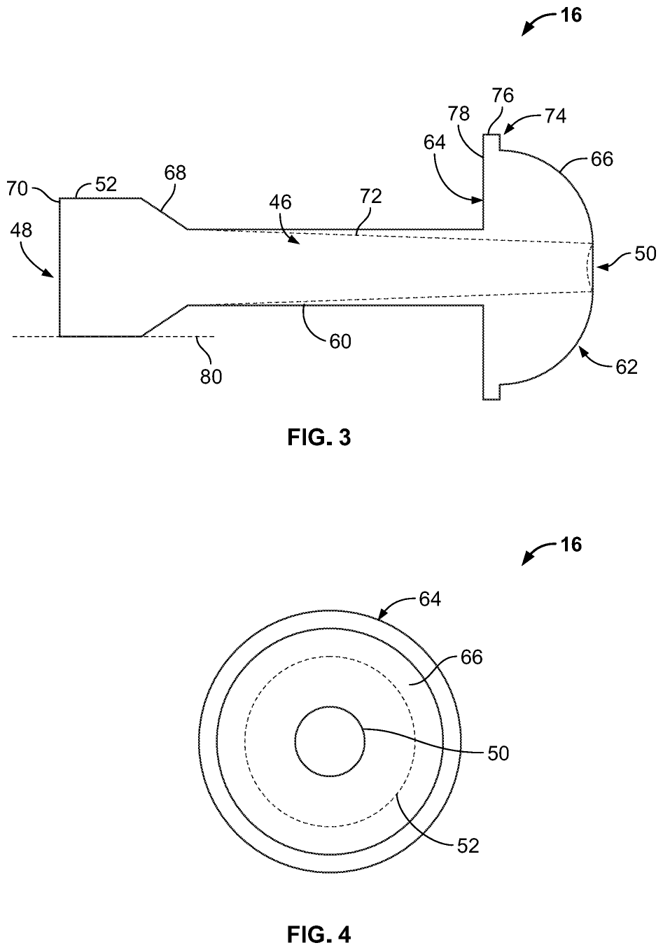

As shown in FIGS. 2-4, the first opening 48 of the prosthetic stoma 16 has a tapered portion 68 that tapers downward toward a cylindrical shaft 60 having a smaller diameter than the first opening 48. The first opening 48 and, thus, the peripheral wall 52 each are defined by a larger diameter than the cylindrical shaft 60. The tapered portion 68 from the peripheral wall 52 may taper toward the shaft 60 at an angle of between 0 and 90 degrees, or about 45 degrees from an axis 80 (see FIG. 3) that extends perpendicularly from an end wall 70 of the prosthetic stoma 16. The cylindrical shaft 60 is hollow (i.e., the dashed lines in FIGS. 2-4 illustrate the hollow passage 46 through the prosthetic stoma 16), thus, the shaft 60 is defined by an elongate continuous inner wall 72. The inner wall 72 defines the passage 46 from the small intestine 20, through the stoma 54, and through the adhesive 14. Additionally, the cylindrical shaft 60 extends from the first opening 48 and terminates at a dome structure 62 provided on a terminal end of the prosthetic stoma 16.

As one skilled in the art would appreciate, the cylindrical shaft 60 can be provided in a variety of sizes and lengths. The size and shape of the cylindrical shaft 60 may be unique to a patient. Additionally, it is within the scope of the invention to include embodiments that do not have a cylindrical shaft 60. In such an embodiment, for example, the peripheral wall 52 may directly connect to the dome structure 62.

The dome structure 62 of the prosthetic stoma 16 is defined by a flanged base 64 and a dome 66. The flanged base 64 includes an annular flange 74 that circumscribes the base 64. The dome 66 extends upward and inward from the flange 74 toward the second opening 50. The flange 74 includes an outer wall 76 that is substantially perpendicular to an end wall 78 of the base 64. The end wall 78 is designed to receive the adhesive 14 to releasably secure the prosthetic stoma 16 to the patient. Therefore, the adhesive 14 can adhere to the end wall 78 of the base 64. Further, the second opening 50 is substantially centered within the dome 66 (see FIG. 4). The second opening 50 is the exit for bodily fluids to evacuate the prosthetic stoma 16 and enter the ostomy bag 12.

Referring again to FIG. 2, a first side 14a of the adhesive 14 is adhered to the ostomy bag 12 and is used to attach the ostomy bag 12 to the patient's abdominal wall 40. As noted above, a second side 14b of the adhesive adheres the base 64 of the prosthetic stoma 16, to the ostomy bag 12, and by extension, to the user's abdominal wall 40. Therefore, the adhesive 14 provides a substantially water-tight seal between the base 64 of the prosthetic stoma 16 and the abdominal wall 40 of the patient. Further, it is within the scope of this disclosure to include the adhesive 14 as an integral component of the prosthetic stoma 16. For example, the adhesive 14 could be a part of the flanged base 64 of the prosthetic stoma 16 and the first side 14a thereof may be applied to the skin of the abdominal wall 40.

As previously noted, the present embodiment of the prosthetic stoma 16 is constructed from a flexible medical grade silicone. However, as one skilled in the art would appreciate, the prosthetic stoma 16 can be made from a number of materials that allow for the transfer of bodily waste from the ostomy to the ostomy bag without causing infection, irritation, and such to the ostomy or other areas of the patient. Additionally, the prosthetic stoma 16 can be used as an additional component to many existing ostomy devices to provide a seal around the user's stoma and reduce or eliminate leakage from the stoma or ostomy bag and should not be limited to the above described examples.

One or more parts of the prosthetic stoma 16 can be provided as a part of, or separate from, the ostomy device 10. In the instance that the prosthetic stoma 16 is provided as a part of the ostomy device 10, the prosthetic stoma 16 may be connected to the adhesive 14 and the ostomy bag 12. Further, in this example, the first side 14a of the adhesive 14 may have a non-sticky backing. During application, the user may insert the first opening 48 and/or the peripheral wall 52 into the artificial stoma 54 and ultimately into the small intestine 20. As a result, the peripheral wall 52 creates a fluid-tight seal between the first opening 48 and the small intestine 20, and the cylindrical shaft 60 creates a fluid-tight seal with the artificial stoma 54. However, the user stops insertion when the end wall 78 of the base 64 is adjacent the user's abdominal wall 40, i.e., a distance of 20 millimeters or less from the abdominal wall. When the end wall 78 is adjacent the user's abdominal wall, the user removes the non-sticky backing from the first side 14a of the adhesive 14 and exposes a sticky area thereon. Accordingly, the user then fully inserts the prosthetic stoma 16, positions the first side 14a of the adhesive 14 flush with the user's abdominal wall 40, and applies a slight force to the prosthetic stoma 16 to adhere the first side 14a of the adhesive 14 to the user's abdominal wall 40.

As previously mentioned, the prosthetic stoma 16 may also be provided separate from the ostomy device 10. The prosthetic stoma 16 is designed to be used with many existing ostomy devices to facilitate drainage of waste products through a surgically created stoma, such as the artificial stoma 54, and into a pouch device similar to the ostomy bag 12. In an instance where the prosthetic stoma 16 is provided separate from the ostomy device 10, a user inserts the first opening 48 or the peripheral wall 52 into the artificial stoma 54. The user then continues to insert the prosthetic stoma 16 until the peripheral wall 52 provides a fluid-tight seal with the small intestine 20 and the cylindrical wall 60 provides a fluid-tight seal with the artificial stoma 54. However, the user stops insertion when the end wall 78 of the base 64 is adjacent the user's abdominal wall 40.

In some embodiments, the user may attach the ostomy device 10, including the adhesive 14 and the ostomy bag 12, to the prosthetic stoma 16. In this example, the adhesive 14 may have a non-sticky backing on each of the first side 14a and the second side 14b of the adhesive 14. First, the user removes the non-sticky backing on the second side 14b, thereby exposing a sticky material thereon and applies the second side 14b to the end wall 78 of the base 64 of the prosthetic stoma 16. As a result, the prosthetic stoma 16 becomes attached to the adhesive 14 and the ostomy bag 12. Once the prosthetic stoma 16 is attached to the ostomy device 10, the non-sticky backing on the first side 14a of the adhesive 14 can be removed. The user then inserts the prosthetic stoma 16 until the first side 14a of the adhesive 14 is adhered to the user's abdominal wall 40. In this particular embodiment, if the user wishes to remove the ostomy device 10 in order to empty the ostomy bag 12 or to replace either the ostomy bag 12 or the adhesive 14, the user can re-use the prosthetic stoma 16 or install a new prosthetic stoma 16.

It will be appreciated that the adhesive used to attach the prosthetic stoma 16 to the user is medical grade.

Additionally, it will be appreciated that there are numerous ways that the prosthetic stoma 16 could be applied. For example, an applicator (not shown) could be provided that may allow the prosthetic stoma 16 to be positioned in an in-use orientation. In one embodiment, the applicator is provided as an elongate cylindrical tube. In this embodiment, one or more protrusions may be provided on a portion of the prosthetic stoma 16 that would act as a catch. For example, one or more protrusions may be provided on the interior of the prosthetic stoma 16 (e.g., within the cylindrical shaft 60) and may be designed to extend at least partially into the hollow passage 46 of the cylindrical shaft 60. In one specific embodiment, four protrusions project inwardly from the wall of the cylindrical shaft 60 toward the hollow passage 46. In use, the prosthetic stoma 16 may be positioned adjacent a patient and the applicator can be aligned with the second opening 50 within the dome 66. The applicator can enter the second opening 50 of the prosthetic stoma 16 and contact the one of more protrusions. In this way, the applicator can be used to facilitate proper placement of the prosthetic stoma 16. It should be appreciated that other applicators and positioning devices may be used to place the prosthetic stoma 16 in an in-use configuration.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. A prosthetic stoma device for use on patients that have undergone ostomy and stoma medical procedures, the prosthetic stoma device comprising:
a first opening, surrounded by a substantially cylindrical peripheral wall, designed to be inserted into a stoma; and
a dome structure including a second opening,
wherein the first opening and the second opening are in communication via a shaft and form a fluid path through the prosthetic stoma device.

2. The prosthetic stoma device of claim 1, wherein the first opening has a tapered portion that tapers downward toward the shaft.

3. The prosthetic stoma device of claim 2, wherein the shaft has a smaller diameter than the first opening.

4. The prosthetic stoma device of claim 1, wherein the shaft is a substantially hollow, cylindrical structure.

5. The prosthetic stoma device of claim 1, wherein the shaft extends from the first opening and terminates at the dome structure.

6. The prosthetic stoma device of claim 5, wherein the dome structure is defined by a flanged base and a dome 66.

7. The prosthetic stoma device of claim 6, wherein the flanged base of the dome structure includes an annular flange that circumscribes the base.

8. The prosthetic stoma device of claim 7, wherein the flange includes an outer wall that is substantially perpendicular to an end wall of the base.

9. The prosthetic stoma device of claim 8, wherein the end wall is designed to receive an adhesive to releasably secure the prosthetic stoma to a patient.

10. A prosthetic stoma kit for use on patients that have undergone ostomy and stoma medical procedures, the prosthetic stoma device kit, comprising:
an ostomy bag;
a prosthetic stoma defined by a shaft having a first opening designed to be positioned adjacent a patient and a second opening provided in a dome structure and designed to be positioned adjacent the ostomy bag; and
an adhesive.

11. The prosthetic stoma kit of claim 10, wherein the stoma is provided as a one piece component.

12. The prosthetic stoma kit of claim 11, wherein at least a portion of the prosthetic stoma is made from an elastically deformable material or flexible medical grade silicone.

13. The prosthetic stoma kit of claim 10, wherein the adhesive is medical grade.

14. The prosthetic stoma kit of claim 10, wherein the adhesive is provided as part of the prosthetic stoma.

15. The prosthetic stoma kit of claim 10 further including an applicator.

16. A prosthetic stoma device, comprising:
a first opening defined by a substantially elastically deformable peripheral wall and a tapered portion; and
an elongate shaft defined by a continuous sidewall that extends from the tapered portion of the first opening and terminates at a dome structure provided on the terminal end of the stoma device and defining a second opening.

17. The prosthetic stoma device of claim 16, wherein the dome structure includes a flanged base having an annular flange that circumscribes the base, and a dome that extends upward and inward from the flange toward the second opening.

18. The prosthetic stoma device of claim 17, wherein the flange includes an outer wall that is substantially perpendicular to an end wall of the base.

19. The prosthetic stoma device of claim 16, wherein the second opening is substantially centered within the dome and the second opening is designed to provide an exit for bodily fluids to evacuate the prosthetic stoma.

20. The prosthetic stoma device of claim 18, wherein the end wall is designed to receive an adhesive to releasably secure the prosthetic stoma to the patient.
